(19) United States
(12) Patent Application Publication
(10) Pub. No.: US 2018/0014797 A1
(43) Pub. Date: Jan. 18, 2018

(54) DIAGNOSTIC OR THERAPEUTIC APPARATUS

(71) Applicant: KUKA Roboter GmbH, Augsburg (DE)

(72) Inventor: Andreas Keibel, Augsburg (DE)

(73) Assignee: KUKA Roboter GmbH

(21) Appl. No.: 15/545,854

(22) PCT Filed: Feb. 8, 2016
(86) PCT No.: PCT/EP2016/052630

§ 371 Date: Jul. 24, 2017

(30) Foreign Application Priority Data
Feb. 10, 2015 (DE) 10 2015 202 327.3

(51) Int. Cl. A61B 6/04; A61N 5/10

(52) U.S. Cl. CPC A61B 6/0407; A61B 6/0457; A61N 2005/1063; A61N 2005/1057

(57) ABSTRACT

A diagnostic or therapeutic apparatus and method for operating the diagnostic or therapeutic apparatus. The diagnostic or therapeutic apparatus includes a stationary diagnostic or therapeutic device, at least one movable patient bed, and at least one manipulator. The patient bed can be coupled to the manipulator in order to enable the manipulator to move the patient bed from a preparation position to a treatment position.

### FIELD OF THE INVENTION

The present invention relates to a diagnostic and/or therapeutic apparatus comprising a diagnostic and/or therapeutic device for diagnosing and/or treating patients, wherein the patients can be moved to the diagnostic and/or therapeutic device by means of at least one patient bed that is coupled to a manipulator. Such a diagnostic and/or therapeutic apparatus can be used, for example, in radiation therapy.

### BACKGROUND

Modern diagnostic and/or therapeutic devices, such as are used, for example, in radiation therapy (e.g., radiation source) or in diagnostics (e.g., CT, MRI), are expensive. Enabling economical operation thereof requires achieving high utilization of the diagnostic and/or therapeutic device. Typically, these diagnostic and/or therapeutic methods require exact positioning of the patient relative to the diagnostic and/or therapeutic device. In order to achieve this, the patients are fixed on a patient bed before the treatment (i.e. before the diagnosis and/or therapy). This fixation is time-consuming, making it impossible to achieve high utilization of the diagnostic and/or therapeutic device if the fixation takes place in the treatment room itself, as is often the case.

The present invention addresses the problem of improving the utilization of a diagnostic and/or therapeutic device and the accuracy of positioning of the patient in the diagnostic and/or therapeutic device.

### DETAILED DESCRIPTION OF THE INVENTION

The aforementioned problem is solved by a diagnostic and/or therapeutic apparatus according to claim 1. In particular, the aforementioned problem is solved by a diagnostic and/or therapeutic apparatus comprising a stationary diagnostic and/or therapeutic device, at least one first movable patient bed, and at least one first manipulator, wherein the first patient bed can be coupled to the first manipulator, and the first manipulator is adapted to move the first patient bed from a preparation position into a treatment position, wherein the treatment position is a position that enables the diagnosis and/or therapy by means of the diagnostic and/or therapeutic device.

The ability to move the patient bed makes it possible to prepare a first patient for the treatment in a preparation position regardless of the current status of the diagnostic and/or therapeutic device. For example, at the point in time that the first patient is being prepared, the diagnostic and/or therapeutic device may be occupied by another patient, or may be prepared for treating the first patient. The spatial separation of preparation and treatment of a patient makes it possible to reduce the length of time that the individual patient occupies the diagnostic and/or therapeutic device, and thus to increase the utilization.

It is advantageous for the patient bed to be moved from the preparation position to the treatment position by means of a manipulator. The at least one manipulator is preferably an automatically guided multi-purpose manipulator that is equipped with three or more freely-programmable axes of movement and is stationary. The at least one manipulator may be coupled to the patient bed and then precisely position same. Alternatively, the at least one manipulator may be fixedly coupled to the patient bed. Preferably, the positioning accuracy of the patient bed that is achieved is better than 5 mm, especially preferably better than 1 mm, and most preferably better than 0.3 mm.

Defined fixation of the patient on the patient bed makes it possible to exactly position the patient relative to the diagnostic and/or therapeutic device. This is important, for example, when tumors are being treated by means of radiation, because the diagnostic and/or therapeutic device must focus the radiation at the site of the tumor in order to treat the tumor tissue.

Preferably, the diagnostic and/or therapeutic apparatus comprises at least one second manipulator and at least one second patient bed, wherein the second patient bed can be coupled to the second manipulator. The use of at least two manipulators and at least two patient beds makes it possible to prepare and/or treat the patients at the same time according to the number of patient beds used. This allows for higher utilization of the diagnostic and/or therapeutic device. The use of a plurality of patient beds and manipulators is especially advantageous if the treatment time in the diagnostic and/or therapeutic device is shorter than the preparation time for the individual patient.

Preferably, the diagnostic and/or therapeutic device is arranged in a treatment room, and each manipulator is set up in a separate preparation room in such a manner that the manipulator and the patient bed can be moved entirely out of the treatment room. Then, the treatment position is preferably set up in the treatment room, and each preparation position preferably set up in a separate preparation room.

This spatial separation enables undisturbed preparation of a patient to be prepared and undisturbed treatment of a patient to be treated, as well as separate preparation of the diagnostic and/or therapeutic device in order to treat the patient to be treated. In particular, the spatial separation also allows for the combination of the diagnostic and/or therapeutic device with an operating room. In this case, at least one preparation room is an operating room. If, during an operation, diagnostic or therapeutic methods that can be performed by means of the diagnostic and/or therapeutic device are needed, then said method can be performed immediately after or before any other use of the diagnostic and/or therapeutic device. This enables a broader scope of use of the diagnostic and/or therapeutic device.

Preferably, the treatment room and the at least one preparation room can be separated from one another by at least one separation apparatus, and the at least one separation apparatus is preferably radiation-proof. The separation apparatuses—e.g., doors or gates—make it possible to separate the treatment room off from the preparation room or the preparation rooms. This is advantageous because hygiene is thus improved. The separation apparatuses may also shield against sounds that are produced in one of the rooms. Especially preferably, the separation apparatuses are radiation-proof and offer protection against the radiation generated by the diagnostic and/or therapeutic device.

Preferably, at least one of the patient beds has fixing means for fixing a patient in a manner com pliant with diagnosis and/or therapy. The fixing means may comprise mechanical devices, such as belts, straps, or screws (e.g., a Mayfield® clamp). The fixing means may also have elements, such as plastic elements, that are modeled on a contour of the patient. The patient may thus be fixed in a defined position on the patient bed.

Furthermore, the diagnostic and/or therapeutic apparatus comprises at least one monitoring means, wherein the monitoring means is set up to monitor the position of a patient bed in the treatment position. Monitoring the position of the patient bed makes it possible to recognize a deviation between the actual position and the intended position of the patient bed. If the deviation is recognized, the manipulator that is coupled to the patient bed can compensate for the deviation by moving and bringing the actual position of the patient bed into alignment with the intended position. This ensures that the patient is positioned in the diagnostic and/or therapeutic device with the desired accuracy.

The aforementioned problem is also solved by a method for switching a patient bed in the diagnostic and/or therapeutic apparatus, comprising the following method steps:
moving the first patient bed from the first preparation position into the treatment position;
performing the diagnosis and/or therapy by means of the diagnostic and/or therapeutic device;
moving the first patient bed out of the treatment position; and
moving the second patient bed from the second preparation position into the treatment position.

Successively moving a first and second patient bed from a preparation position assigned to the patient beds into the treatment position enables preparation of patients that is spatially and temporally independent of the actual treatment. The method enables the combination of a plurality of patient beds with only one diagnostic and/or therapeutic device, thus allowing for higher utilization of the diagnostic and/or therapeutic device.

Preferably, prior to the movement of the first patient bed from the first preparation position into the treatment position, a first separation device is opened between the treatment position and the first preparation position.

Also, a second separation device, which is arranged between a second preparation position and the treatment room, may be closed before the first patient bed is moved from the first preparation position into the treatment position. This ensures that the patients can be prepared spatially and temporally independently of the actual treatment, while the privacy of the patients is also being respected. If the first patient bed is being moved from the treatment position back into the first preparation position, then—after the first preparation position has again been reached—the first separation device can first be closed before the second separation device is opened.

### DESCRIPTION OF THE DRAWINGS

Preferred embodiments of the present invention are described in further detail hereinbelow, with reference to the drawings. In the drawings,

FIG. 1 illustrates a diagnostic and/or therapeutic apparatus, wherein a first patient bed is in the treatment position, and

FIG. 2 illustrates a diagnostic and/or therapeutic apparatus, wherein a second patient bed is in the treatment position.

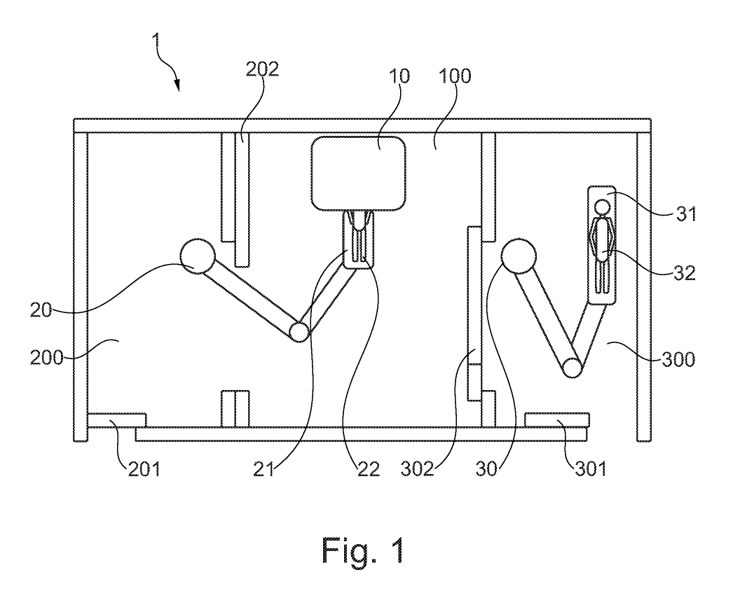

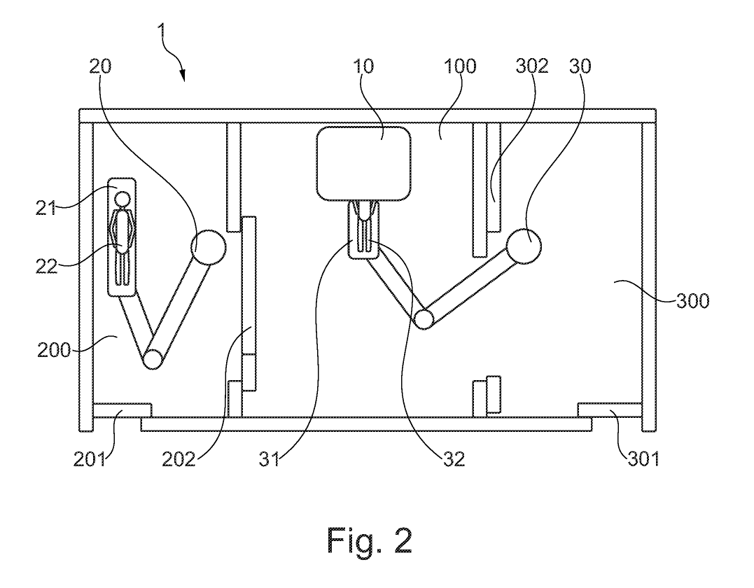

FIGS. 1 and 2 illustrate a diagnostic and/or therapeutic apparatus 1 comprising a diagnostic and/or therapeutic device 10, a first and second manipulator 20, 30, and a first and second patient bed 21, 31. The first manipulator 20 is coupled to the first patient bed 21 in the depiction illustrated. Also, the second manipulator 30 is coupled to the second patient bed 21. A first patient 23 is on the first bed 21, and a second patient 32 is on the second bed 31.

The first manipulator 20 is set up in a first preparation room 200 and the second manipulator 30 is set up in a second preparation room 300. The two preparation rooms 200, 300 are next to a treatment room 100 in which the diagnostic and/or therapeutic device is arranged. The first preparation room 200 can be separated off from the treatment room 100 by a first separation device 202. As illustrated, the first separation device is, for example, a sliding door. The sliding door can be operated preferably automatically or manually. The preparation rooms 200, 300 can be entered from the outside through doors 201, 301. Thus, the patient need not pass through the treatment room in order to reach the preparation room.

As illustrated in FIG. 1, the first patient bed 21 can be moved by means of the first manipulator 20 from a preparation position in the preparation room 200 into the treatment position in the diagnostic and/or therapeutic device 10 in the treatment room 100. The second manipulator 30 illustrated in FIG. 1 and the second patient bed 31 have been entirely withdrawn into the second preparation room 300. A separation apparatus 302 separates the preparation room 300 off from the treatment room 100. Thus, the second patient 32 can be prepared while the first patient is being treated.

FIG. 2 illustrates the diagnostic and/or therapeutic apparatus 1 of FIG. 1, wherein, in the depiction illustrated, the first manipulator 20 and the first patient bed 21 have been entirely withdrawn into the first preparation room 200 and the patient bed 31 is located in the treatment position. Thus, follow-up with a first patient 22 can be performed while the second patient 32 is being treated, or a third patient (not shown) can be prepared without disturbing the course of treatment of the second patient.

### REFERENCE NUMERALS

1 . . . Diagnostic and/or therapeutic apparatus
10 . . . Diagnostic and/or therapeutic device
20 . . . First manipulator
21 . . . First patient bed
22 . . . First patient
30 . . . Second manipulator
31 . . . Second patient bed
32 . . . Second patient
100 . . . Treatment room
200 . . . First preparation room
201 . . . First door
202 . . . First separation device
300 . . . Second preparation room
301 . . . Second door
302 . . . Second separation device

1. A diagnostic or therapeutic apparatus comprising:
a stationary diagnostic and/or therapeutic device,
at least one first movable patient bed, and
at least one first manipulator, wherein:
the first patient bed is configured be coupled to the first manipulator,
the first manipulator is set up to move the first patient bed from a preparation position to a treatment position, and
the treatment position is a position that enables the diagnosis or therapy by means of the diagnostic or therapeutic device.

2. The diagnostic or therapeutic apparatus according to claim 1, comprising at least one second manipulator and at least one second patient bed, wherein the second patient bed is configured to be coupled to the second manipulator.

3. The diagnostic or therapeutic apparatus according to claim 1, wherein the diagnostic or therapeutic device is arranged in a treatment room, and each manipulator is set up in a separate preparation room in such a manner that the manipulator and the patient bed can be moved entirely out of the treatment room.

4. The diagnostic or therapeutic apparatus according to claim 1, wherein the treatment position is set up in the treatment room and each preparation position is set up in a separate preparation room.

5. The diagnostic or therapeutic apparatus according to claim 1, wherein the treatment room and the at least one preparation room can be separated from one another by at least one separation apparatus, and the at least one separation apparatus is radiation-proof.

6. The diagnostic or therapeutic apparatus according to claim 1, wherein at least one of the patient beds includes fixing means for fixing a patient in a manner conforming with the diagnosis and/or therapy.

7. The diagnostic or therapeutic apparatus according to claim 1, wherein the diagnostic or therapeutic apparatus includes at least one monitoring means, wherein the monitoring means is set up to monitor the position of a patient bed in the treatment position.

8. A method for switching a patient bed in the diagnostic or therapeutic apparatus, comprising a stationary diagnostic and/or therapeutic device, at least one first movable patient bed, a second movable patient bed and at least one first manipulator, the method comprising the steps of:
moving the first movable patient bed by the first manipulator from a first preparation position to a treatment position;
performing the diagnosis or therapy by means of the diagnostic or therapeutic device;
moving the first movable patient bed by the first manipulator out of the treatment position; and
moving the second movable patient bed by the first manipulator from a second preparation position into the treatment position.

9. The method according to claim 8, wherein a first separation device is opened between the treatment position and the first preparation position prior to the movement of the first patient bed from the first preparation position into the treatment position.

10. The diagnostic or therapeutic apparatus according to claim 2, wherein the diagnostic and/or therapeutic device is arranged in a treatment room, and each manipulator is set up in a separate preparation room in such a manner that the manipulator and the patient bed can be moved entirely out of the treatment room.

11. The diagnostic or therapeutic apparatus according to according to claim 2, wherein the treatment position is set up in the treatment room and each preparation position is set up in a separate preparation room.

12. The diagnostic or therapeutic apparatus according to according to claim 2, wherein the treatment room and the at least one preparation room can be separated from one another by at least one separation apparatus, and the at least one separation apparatus is radiation-proof.

13. The diagnostic or therapeutic apparatus according to according to claim 2, wherein at least one of the patient beds includes fixing means for fixing a patient in a manner conforming with the diagnosis and/or therapy.

14. The diagnostic or therapeutic apparatus according to according to claim 2, wherein the diagnostic or therapeutic apparatus includes at least one monitoring means, wherein the monitoring means is set up to monitor the position of a patient bed in the treatment position.

15. The diagnostic or therapeutic apparatus according to claim 3, wherein the diagnostic and/or therapeutic device is arranged in a treatment room, and each manipulator is set up in a separate preparation room in such a manner that the manipulator and the patient bed can be moved entirely out of the treatment room.

16. The diagnostic or therapeutic apparatus according to according to claim 3, wherein the treatment position is set up in the treatment room and each preparation position is set up in a separate preparation room.

17. The diagnostic or therapeutic apparatus according to according to claim 3, wherein the treatment room and the at least one preparation room can be separated from one another by at least one separation apparatus, and the at least one separation apparatus is radiation-proof.

18. The diagnostic or therapeutic apparatus according to according to claim 3, wherein at least one of the patient beds includes fixing means for fixing a patient in a manner conforming with the diagnosis and/or therapy.

19. The diagnostic or therapeutic apparatus according to according to claim 3, wherein the diagnostic or therapeutic apparatus includes at least one monitoring means, wherein the monitoring means is set up to monitor the position of a patient bed in the treatment position
